(19) United States
(12) Patent Application Publication
(10) Pub. No.: US 2010/0274085 A1
(43) Pub. Date: Oct. 28, 2010

(54) EXCHANGEABLE GUIDE-WIRE WITH BALLOON FOR FOREIGN BODY EXTRACTION

(76) Inventors: John Mugan, Galway (IE); Brian Murphy, Galway (IE); John McWeeney, Brighton, MA (US)

Correspondence Address: BROWN RUDNICK LLP, ONE FINANCIAL CENTER, BOSTON, MA, 02111, US

(21) Appl. No.: 12/810,324

(22) PCT Filed: Dec. 29, 2008
(86) PCT No.: PCT/US08/88431

§ 371 Date: Jun. 24, 2010

Related U.S. Application Data
(60) Provisional application No. 61/017,489, filed on Dec. 28, 2007.

(51) Int. Cl. A61B 1/04

(52) U.S. Cl. 600/115

(57) ABSTRACT

A guide-wire device is presented. The guide-wire device is includes a guide-wire housing, an inner shaft member disposed within the guide-wire housing, and a balloon for use in foreign body extraction and as an anchoring means to facilitate the exchange of ancillary devices over the guide-wire housing. An inflation hub is adapted to form a coupling with the proximal portion of the guide-wire housing and a flexible tip is disposed to the distal portion of the balloon.

### CROSS-REFERENCE TO RELATED APPLICATIONS

This patent application claims priority to U.S. Provisional Application Ser. No. 61/017,489, filed in the U.S. Patent and Trademark Office on Dec. 28, 2007 by McWeeney et al, the contents of that application being incorporated herein by reference in its entirety.

### BACKGROUND

1. Technical Field

The present disclosure generally relates to the field of guide-wire devices, and more particularly, to guide-wire devices having a balloon for foreign body extraction.

2. Description of the Related Art

Endoscopic retrograde cholangiopancreatography (ERCP) is a technique that combines the use of endoscopy and fluoroscopy to diagnose and treat certain ailments of the biliary or pancreatic ductal systems. ERCP involves the use of a video endoscope and the x-ray examination of a patient's bile ducts. For example, a user may visualize the inside of a patient's stomach and duodenum through an endoscope and, subsequently, inject dyes into the patient's bile and pancreatic ducts in the biliary tree for x-ray examination with a view to therapeutic selection.

ERCP may be performed for diagnostic applications, such as, obstructive jaundice, chronic pancreatitis, gallstones with dilated bile ducts on ultrasonography, bile duct tumors, suspected injury to bile ducts as a result of trauma, and sphincter of oddi dysfunction. ERCP may also be performed for therapeutic applications, such as, endoscopic sphincterotomy (both of the biliary and the pancreatic sphincters), removal of stones, insertion of stent(s), and the dilation of strictures (e.g., primary sclerosing, choloangitis, anastomotic strictures after liver transplantation, and the like).

Typical ERCP procedures begin with a user advancing an endoscope trans-orally through a patient's esophagus, across the pylorus, and into the duodenum. A tome catheter, cannula, or papillatome is then inserted through the ampulla of vater and contrast medium is injected into the bile ducts and/or pancreatic duct. Subsequently, fluoroscopy is used to examine the patient's biliary tree for blockages or leakage of bile into the peritoneum (the abdominal cavity) and the like.

Sphincterotomy procedures are also performed during ERCP via the use of a tome with cutting wire. A sphincterotomy involves the action of energizing the cutting wire of the tome to cut a sphincter to allow the passage of larger diameter therapeutic devices. These therapeutic devices may be, for example, stents and stone retrieval devices to remedy biliary tree conditions. Subsequently, a guide-wire with a diameter in the range of approximately 0.020-0.038 inches is inserted through the tome catheter and is tracked into the pancreatic or common bile ducts to a desired location. The guide-wire serves as a tracking medium during the remainder of the ERCP procedure to guide stents, dilatation balloons, and stone extraction devices to a desired location in the biliary ductal system.

A majority of ERCP procedures involve the management of stones in a patient's biliary tree. Presently, multiple devices and/or multiple exchanges of devices are required to adequately treat stones in a patient's common bile duct. For example, the treatment of stones in a common bile duct may require the removal of a guide-wire on multiple instances to insert other devices, such as stone retrieval balloons or stone retrieval baskets. As a result, the need for multiple exchanges in advancing devices over a “free-floating” guide-wire requires extreme caution and a high level of skill on the part of the user to maintain guide-wire position in the biliary ductal system. Additionally, the need for multiple exchanges prolongs the average ERCP procedure time, increases fluoroscope exposure, increases a patient's sedation time, and reduces procedural efficiency.

The current technological practice for stone removal from a patient's biliary ductal system involves the following need(s): (i) for a user to maintain guide-wire access in the patient's duct; (ii) to feed a dilation balloon product over the guide-wire device; (iii) to inflate the balloon; (iv) to perform “sweeps” of the ductal system to “drag” the foreign object(s) in question across the ampulla of vater; and (v) to deposit the foreign object(s) into the duodenum for natural excretion through the remainder of the alimentary canal. In such instances, there is a danger that a user may lose access across the ampulla leading to prolonged procedures or the potential abandonment of a procedure.

Traditional guide-wire devices are formed from a continuous length of wire (typically stainless steel), which is helically wound or coiled along the longitudinal axis of a core wire. The continuous length of wire provides a finished device with an outer diameter of approximately 0.020 to 0.038 inches. Guide-wire devices of this nature may be uncoated or may be coated with a polymer to enhance the lubricity of the surface, such as polytetrafluoroethylene or TEFLON®. These guide-wires devices, however, suffer from several drawbacks. For example, such devices suffer from poor resistance to kinking under conditions of tortuosity. As a result, the guide-wire of such devices must be exchanged for another guide-wire, thereby causing the user to loose access to a patient's ampulla of vater or ductal system. Additionally, the need for a user to re-access a patient's ductal system can be very cumbersome or cannot be achieved resulting in prolonged or abandoned procedures.

Guide-wire devices with integrated balloon technology have been widely used in the field of coronary angioplasty for dilating stenotic vascular lesions in the coronary vasculature. These devices, however, are not intended for use as an over-the-wire exchange mechanism to exchange catheters, endoscopes, or other devices thereover. For example, the removal of an integrated device's inflation hub or inflation device can result in the inadvertent deflation of its balloon, the potential loss of position, and/or access for the user. The adoption of such guide-wire devices with integrated balloon technology for the field of ERCP would likewise suffer similar drawbacks in the event that the pressure is removed from the system. Therefore, a need exists for an improved exchangeable guide-wire device having a balloon for the extraction of foreign objects.

### SUMMARY

According to an aspect of the present disclosure, a guide-wire device is presented. The guide-wire device includes a guide-wire housing having proximal and distal portions, an inner shaft member disposed within the guide-wire housing, a balloon for use in foreign body extraction, an inflation hub disposed to the proximal portion of the guide-wire housing, and a flexible tip disposed to the distal portion of the balloon. The inner shaft member of the guide-wire device extends from the proximal portion of the guide-wire housing through the distal portion of the guide-wire housing. The balloon of the guide-wire device is disposed to the distal portion of the guide-wire housing. The inflation hub of the guide-wire device includes a means to inflate and deflate the balloon. The flexible tip of the guide-wire device provides a housing to a core comprised of kink-resistant materials.

According to another aspect of the present disclosure, a guide-wire device is presented. The guide-wire device includes a guide-wire housing having proximal and distal portions, an inner shaft member disposed within the guide-wire housing, a balloon for use as an anchoring means to facilitate the exchange of ancillary devices over the guide-wire housing, an inflation hub disposed to the proximal portion of the guide-wire housing, and a flexible tip disposed to the distal portion of the balloon. The inner shaft member of the guide-wire device extends from the proximal portion of the guide-wire housing through the distal portion of the guide-wire housing. The balloon of the guide-wire device is disposed to the distal portion of the guide-wire housing. The inflation hub of the guide-wire device includes a means to inflate and deflate the balloon. The flexible tip of the guide-wire device provides a housing to a core comprised of kink-resistant materials.

According to yet another aspect of the present disclosure, a guide-wire device is presented. The guide-wire device includes a guide-wire housing having proximal and distal portions, an inner shaft member disposed within the guide-wire housing, a balloon for use in foreign body extraction and as an anchoring means to facilitate the exchange of ancillary devices over the guide-wire housing, an inflation hub adapted to form a coupling with the proximal portion of the guide-wire housing, and a flexible tip disposed to the distal portion of the balloon. The inner shaft member includes a proximal inner shaft, a middle inner shaft, a distal inner inflation shaft, and an inflation check valve. The inner shaft member also extends from the proximal portion of the guide-wire housing through the distal portion of the guide-wire housing. The balloon of the guide-wire device is disposed to the distal portion of the guide-wire housing. The inflation hub of the guide-wire device is disposed to the proximal portion of the guide-wire housing and includes a means to inflate and deflate the balloon. The flexible tip of the guide-wire device provides a housing to a core comprised of kink-resistant materials.

### BRIEF DESCRIPTION OF THE DRAWINGS

The objects and features of the present disclosure, which are believed to be novel, are set forth with particularity in the appended claims. The present disclosure, both as to its organization and manner of operation, together with further objectives and advantages, may be best understood by reference to the following description, taken in connection with the accompanying drawings as set forth below:

FIG. 1 is a perspective view of a guide-wire device having a balloon, according to the present disclosure;

FIG. 2 is a perspective view of another embodiment of a guide-wire device having a balloon, according to the present disclosure;

FIG. 3 is a perspective view of an embodiment of an inflation hub of a guide-wire device having a balloon, according to the present disclosure;

FIG. 4 is a perspective view of an embodiment of the distal portion of a guide-wire device having a balloon, according to the present disclosure;

FIG. 4a is a perspective view of an embodiment of an inner shaft member of guide-wire device having a balloon, according to the present disclosure;

FIG. 4b is a perspective view of another embodiment of an inner shaft member of a guide-wire device having a balloon, according to the present disclosure;

FIG. 4c is a perspective view of another embodiment of an inner shaft member of a guide-wire device having a balloon, according to the present disclosure;

FIG. 4d is a perspective view of another embodiment of an inner shaft member of a guide-wire device having a balloon, according to the present disclosure;

FIG. 5 is a perspective view of an embodiment of the proximal portion of a guide-wire device having a balloon, according to the present disclosure;

FIG. 6a is a perspective view of an embodiment of an inflation hub of a guide-wire device having a balloon, according to the present disclosure;

FIG. 6b is a perspective view of another embodiment of an inflation hub of a guide-wire device having a balloon, according to the present disclosure;

FIG. 6c is a perspective view of yet another embodiment of an inflation hub of a guide-wire device having a balloon, according to the present disclosure;

FIG. 7 is a perspective view of an embodiment of an inflation check valve of a guide-wire device having a balloon, according to the present disclosure;

FIG. 8 is a perspective view of an inflation check valve of a guide-wire device having a balloon, according to the present disclosure;

FIG. 9 is a perspective view of another embodiment of an inflation check valve of a guide-wire device having a balloon, according to the present disclosure;

FIG. 10 is a cross-sectional view of an another embodiment of an inflation check valve of a guide-wire device having a balloon, according to the present disclosure;

FIG. 11 is a perspective view of another embodiment of an inflation check valve of a guide-wire device having a balloon, according to the present disclosure; and

FIG. 12 illustrates an embodiment of a guide-wire device having a balloon in an example field of use, according to the present disclosure.

### DETAILED DESCRIPTION OF EXEMPLARY EMBODIMENTS

The present disclosure generally relates to the field of guide-wire devices, and more particularly, to guide-wire devices having a balloon for foreign body extraction.

In the discussion that follows, the term “proximal” refers to a portion of the guide-wire device that is closer to a user, and the term “distal” refers to a portion that is farther from the user. According to the present disclosure, the term “user” refers to an individual performing foreign body extraction or exchanging of ancillary devices over the guide-wire device, installing or removing an inflation hub from an exchangeable guide-wire device, and may include support personnel.

Reference will now be made in detail to exemplary embodiments of the present disclosure, which are illustrated in the accompanying figures. The same reference numbers in different drawings may identify the same or similar elements. In addition, the following detailed description does not limit the present disclosure.

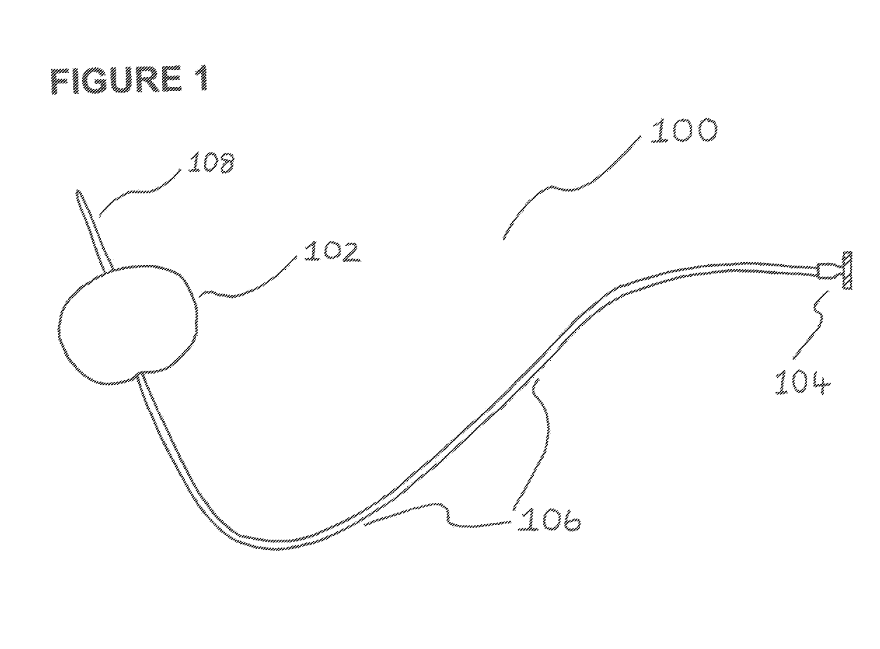

Referring to FIG. 1, a guide-wire device 100 having a balloon for foreign body extraction is presented. Guide-wire device 100 is generally comprised of a balloon 102, an inflation hub 104, a guide-wire housing 106, a valve mechanism (not shown in Figure), and a tip 108. In an embodiment, the outer diameter of guide-wire device 100 does not exceed 0.038 inches over its length in order to facilitate exchange and compatibility with ancillary medical devices used during typical ERCP procedures.

Balloon 102 is positioned on the distal portion of guide-wire device 100. Balloon 102 may inflate to a diameter greater than the diameter of the duct into which it is inserted. The ability to inflate balloon 102 to such a diameter can provide sufficient interference such that guide-wire device 100 is secure in position and does not inadvertently displace during manipulation and subsequent device passage. The operability of balloon 102 permits guide-wire device 100 to be used as, for example, a “stone extraction” mechanism to remove stones and debris from the common bile or pancreatic ducts.

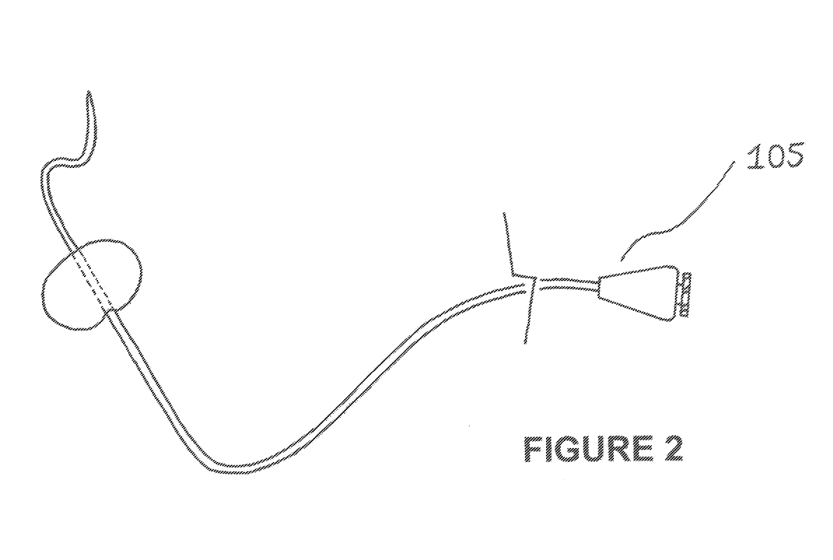

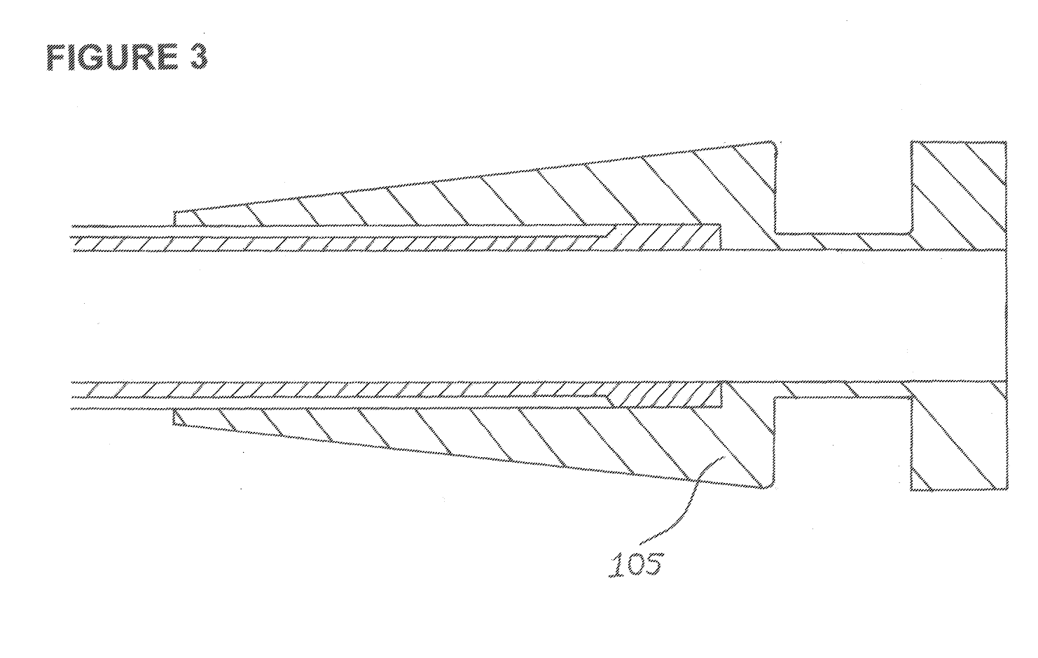

Inflation hub 104 is located on the proximal portion of guide-wire device 100. Inflation hub 104 may be, for example, detached from the body of guide-wire housing 106. Referring now to FIGS. 2 and 3, an embodiment of an inflation hub 105 of guide-wire device 100 is presented. Inflation hub 105 may be non-detachable or exchangeable. The proximal end of guide-wire device 100 may incorporate a permanently over-molded hub/luer feature, which is used to attach to an external inflation mechanism, such as a syringe or inflation pump. Once balloon 102 has reached its required amount of inflation, the inflation is ceased, and balloon 102 remains inflated via the constant application of pressure via a syringe or inflation mechanism.

A user utilizing guide-wire device 100 may begin an ERCP procedure by initially cannulating a patient's common bile or pancreatic ducts, and subsequently performing a cholangiogram. In the present example, the user may insert guide-wire device 100 pre or post contrast injection, advance guide-wire device 100 to a desired location in the patient's biliary tree, inflate balloon 102, and perform stone and foreign body extraction by sweeping the patient's duct.

Referring now to FIG. 1, guide-wire housing 106 may be a solid metallic or polymeric structure. Guide-wire housing 106 may also be a coiled configuration which encapsulates a lumen through which the inflation medium for balloon 102 is injected or aspirated. The valve mechanism (not shown) of guide-wire device 100 is located at the proximal portion of guide-wire housing 106. The valve mechanism may facilitate the inflation of balloon 102 via an inner lumen encased in the body of guide-wire 106, the maintenance of inflation pressure of balloon 102, and the deflation of balloon 102 as desired by the user at such time as guide-wire device 100 needs to be removed or re-positioned. The position of the valve mechanism may provide, for example, an injection port for inflation and deflation of balloon 102 and a flow control valve (also known as an inflation gasket). The flow control valve of guide-wire device 100 shall allow for the removal of the inflate and deflate mechanism to facilitate the rapid exchange of devices over the body of guide-wire 106.

The distal portion of guide-wire device 100, which is distal to balloon 102, is provided as tip 108. Tip 108 facilitates the passage of balloon 102 and guide-wire housing 106 past foreign objects such as, stones or strictures, to the intra-hepatic ducts in a patient's biliary tree. The outer-surface of tip 108 may be of a low coefficient of friction for the ease of passage and tracking of guide-wire device 100. Further, tip 108 may facilitate a means for a user to maintain ductal access during the extraction of foreign bodies from a patient's ductal system.

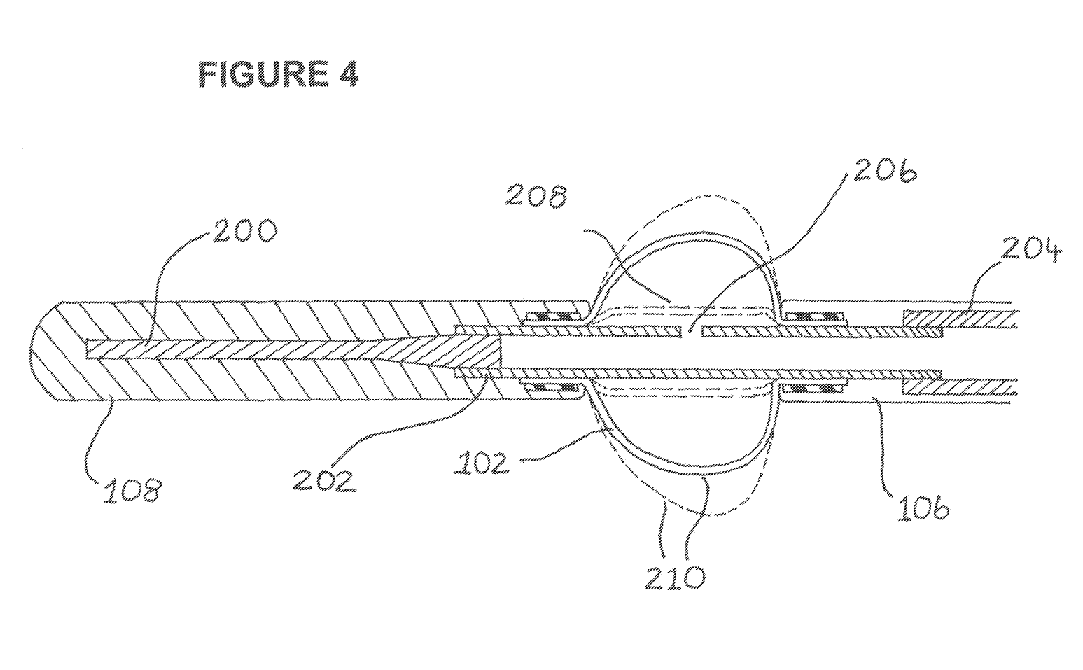

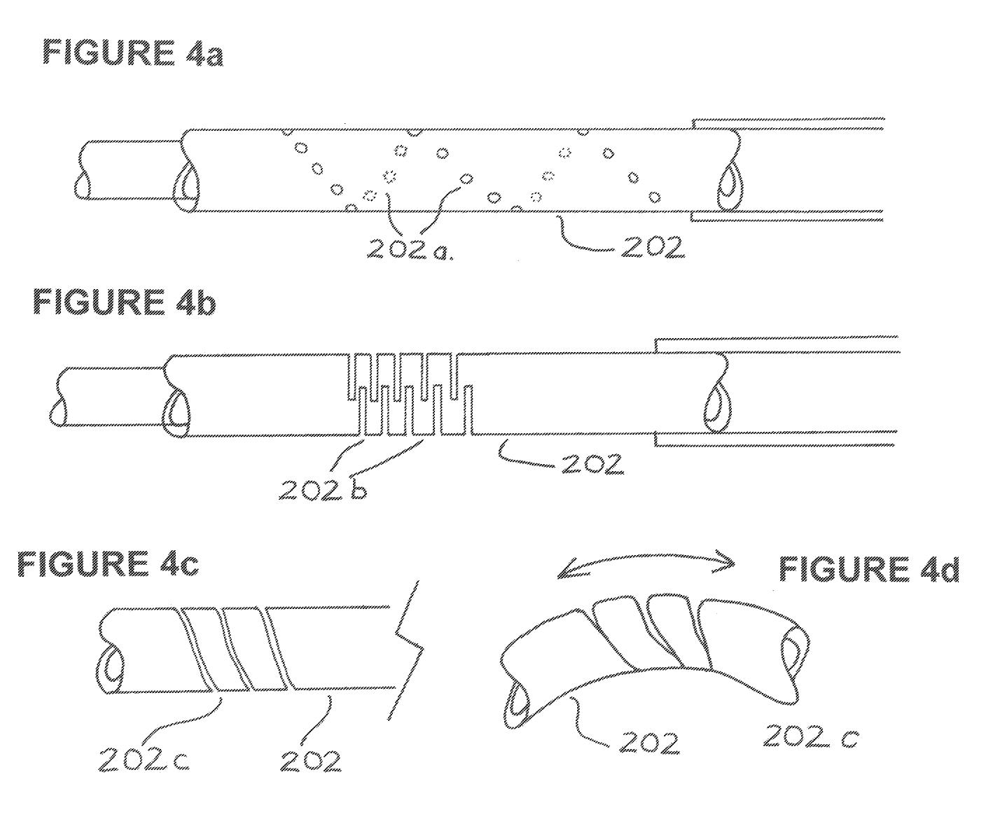

Referring to FIG. 4, a perspective view of an embodiment of the distal portion of guide-wire device 100 is presented. The distal portion of guide-wire device 100 is generally comprised of balloon 102, tip 108, a tip core wire 200, a distal inner inflation shaft 202, a middle inner shaft 204, and an inflation aperture 206.

Tip 108 may be 2 centimeters (cm) to 25 centimeters (cm) in length; however, a length of 5 to 10 cm may be desirable. Tip 108 provides a housing for tip core wire 200 and may be kink-resistant and tapered across its length to provide a stiffness transition from the distal to the proximal end. The tapered geometry of tip core wire 200 may be achieved via center-less grinding, necking, or cold forming a solid piece of wire. Tip core wire 200 may be manufactured of a kink-resistant metallic based material, such as, but not limited to, nitinol or derivatives thereof.

Tip core wire 200 may be solid in nature and tapered from the distal to proximal end. In an embodiment of the present disclosure, the diameter of tip core wire 200 is between 0.005-0.025 inches so that tip 108 will not kink whilst being tracked past tight strictures or large impacted stones in biliary, intra-hepatic, or pancreatic ductal segments.

Tip core wire 200 may be encased in a polymeric covering to provide for atraumatic advancement during insertion. The polymeric covering of tip 108 may be comprised of thermoplastics such as polyurethane, polyamide, polyether-block amide or co-polymers thereof, polyethylene or derivatives thereof, polyvinyl chloride, or any combination thereof. Tip 108 may be filled with a medium, such as barium, barium sulfate, bismuth subcarbonate, bismuth oxychloride, tungsten, or any combination thereof, to enhance the radiopacity of tip 108. As a result, tip 108 may be readily visible under fluoroscopy.

The polymeric material of tip 108 may be bonded onto core wire tip 200 via thermal or adhesive bonding techniques. In an embodiment, tip 108 is lubricous in nature so as to improve trackability through a patient's ductal system. The lubricity of tip 108 may be improved via coating with a hydrophilic polymer or derivatives thereof.

Core Wire tip 200 is attached proximally to distal inner inflation shaft 202. Distal inner inflation shaft 202 may be a tubular structure, which is preferably metallic in nature. Distal inner inflation shaft 202 may be manufactured from a metal such as, stainless steel, nickel, nickel-titanium or alloys thereof, cobalt, chromium or alloys thereof, alloys, or any combination thereof. In an embodiment, distal inner inflation shaft 202 is manufactured from nitinol or derivatives thereof.

Distal inner inflation shaft 202 resides beneath balloon 102 and is distal to a middle inner shaft 204. Distal inner inflation shaft member 202 may be bonded distally to core wire tip 200 via metal joining techniques such as, adhesive, welding, and soldering. Similarly, distal inner inflation shaft 202 may be bonded proximally to middle inner shaft 204 via metal joining techniques such as welding, soldering, or via adhesive bonding. Distal inner inflation shaft 202 incorporates at least one inflation aperture 206. Aperture 206 can serve as the exit point for inflation media, which is injected proximally into guide-wire device 100 to facilitate the inflation of balloon 102.

Referring now to FIGS. 4a through 4d, perspective views of alternative embodiments of distal inner inflation shaft 202 are presented. Inflation aperture 206 of distal inner inflation shaft 202 may be provided in various forms. For example, inflation aperture 206 may be a circular or elliptical structure with hole-drilled or laser-cut apertures through the wall of distal inner inflation shaft 202.

In an embodiment of the present disclosure, distal inner inflation shaft 202 includes a number of apertures or holes 202a that are cut into distal inner inflation shaft 202 in a helical fashion. Holes 202a may also be arranged in a linear fashion along the longitudinal axis of distal inner inflation shaft 202. In another embodiment, holes 202a may be arranged in a circumferential fashion around distal inner inflation shaft 202. It is contemplated that the arrangement of holes 202a in a helical fashion may improve the kink resistance of this component in bending.

In another embodiment of the present disclosure, distal inner inflation shaft 202 includes laser-cut slots 202b. Laser-cut slots 202b may be machined into one or both sides of distal inner inflation shaft 202. The depth of laser-cut slot 202b can overlap the end location of laser-cut slot 202b from the opposing side of distal inner inflation shaft 202 (i.e., the depth of laser-cut slot 202b is greater than the outer radius of proximal inner shaft 300). It is contemplated that the depth of laser-cut slot 202b may prevent distal inner inflation shaft 202 from kinking in flexure during use. In yet another embodiment, distal inner inflation shaft 202 includes spiral cuts 202c. Spiral cuts 202c may enhance the resistance of distal inner inflation shaft 202 to kinking or bending.

Referring now to FIG. 4, balloon 102 may be bonded both proximally and distally to distal inner inflation shaft 202. Balloon 102 may be manufactured from elastomeric, compliant latex or silicone, silicone elastomers, polyurethane or polyurethane elastomers or other materials, which have the ability to expand from a state of pre-inflation 208 to a state of post-inflation 210. The wall thickness of balloon 102 may be, for example, 0.002-0.010 inches.

In an embodiment of the present disclosure, balloon 102 expands from a pre-inflation diameter of 0.020-0.040 inches to diameters of 0.450-0.550 inches, post inflation. In another embodiment, the outer diameter of balloon 102 has a diameter equal to or less than the diameter of guide-wire housing 106 when fully deflated or in the state of pre-inflation 208. It is desirable that in the state of post inflation 210, balloon 102 will elastically return to substantially the same pre-inflated geometry so as to seat against distal inner inflation shaft 202 as close as possible.

Balloon 102 may be attached to middle inner shaft 204 both distal and proximal to the location of inflation aperture 206. Balloon 102 may be “locked” in position via at least one circumferential balloon anchors 212, which are located at the distal and proximal necks of balloon 102. Balloon anchors 212 may take the form of circular stainless steel bands, which are approximately 0.002-0.004 in diameter. Balloon anchors 212 may be crimped onto distal inner inflation shaft 202 to sandwich the distal and proximal necks of balloon 102. The act of crimping maintains an airtight attachment seal of balloon 102 to distal inner inflation shaft 202.

In an embodiment of the present disclosure, balloon 102 may be attached to distal inner inflation shaft 202 via wrapping a suture or thread material around each of the proximal and distal necks of balloon 102. Subsequently, balloon 102 may be attached by applying adhesive to securely bond the thread to balloon 102 and to anchor balloon 102 to distal inner inflation shaft 202. In another embodiment, balloon 102 may be secured to middle inner shaft 204 via thermal bonding techniques. In the present embodiment, polymeric tip 108 encases the distal portion of balloon 102 through a portion of distal inner inflation shaft 202 to provide a smooth transition from balloon 102 to tip 108.

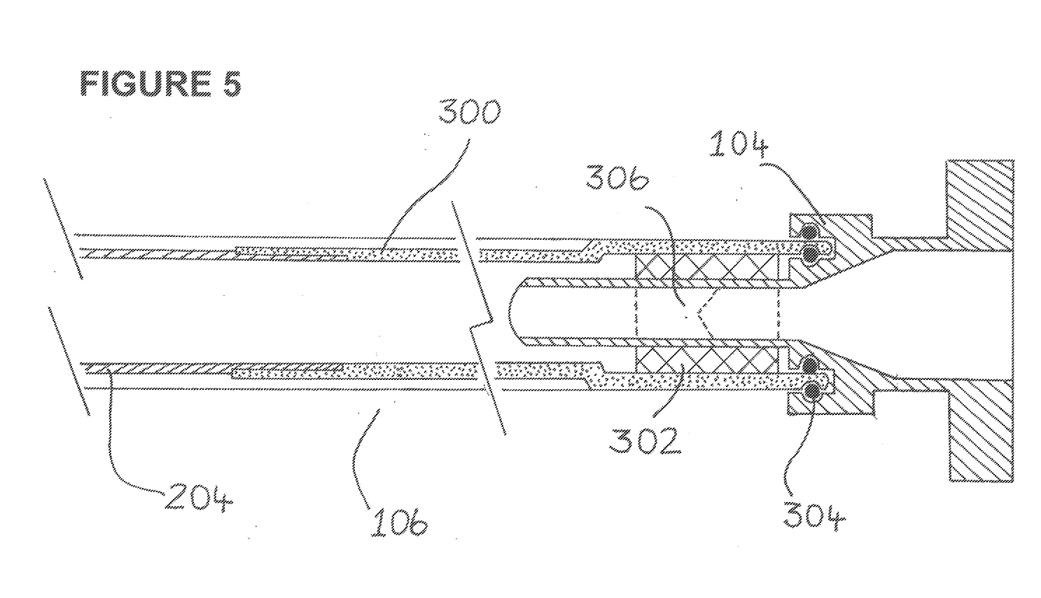

Referring to FIG. 5, a perspective view of an embodiment of the proximal portion of guide-wire device 100 is presented. Guide-wire device 100 may further include a proximal inner shaft 300. The length of guide-wire device 100 from the bond site of middle inner shaft 204 and proximal inner shaft 300 to the proximal end of guide-wire device 100 may be 180 cm to 400 cm. In an embodiment of the present disclosure, the length of guide-wire device 100 with exchangeable inflation hub 104 is approximately 260 cm. In this particular instance, the overall length of guide-wire device 100 may be approximately 220 to 280 cm. In another embodiment, the length of guide-wire device 100 with non-detachable inflation hub 105 is approximately 460 cm. In this instance, the overall length of guide-wire device 100 may be approximately 440 to 500 cm.

Proximal inner shaft 300 and middle inner shaft 204 are encased proximal to balloon 102 by guide-wire housing 106. Guide-wire housing 106 may be comprised of polymeric materials. Guide-wire housing 106 may be provided from the proximal portion of guide-wire device 100 to immediately proximal to balloon 102.

Guide-wire housing 106 may be manufactured from a lubricous polymer or polymeric compound such as, teflon (PTFE) impregnated polyurethane, polyamide, polyether-block amide or co-polymers thereof, polyethylene or derivatives thereof, poly vinyl chloride, fluoronated ethylene propylene (FEP) or polytetrafluoroethylene (PTFE), or any combination thereof. In an embodiment of the present disclosure, Guide-wire housing 106, which is comprised of polymeric materials, may also be coated with a hydrophilic coating to improve the lubricity of guide-wire device 100. Such coating can aid in the passage of guide-wire device 100 over the wire during device exchanges.

Middle inner shaft 204 is adjoined to proximal inner shaft 300. Middle inner shaft 204 may be comprised of metals such as, stainless steel, nickel, titanium, chromium-cobalt, or alloys thereof. In an embodiment of the present disclosure, middle inner shaft 204 is manufactured from nitinol, chromium-cobalt, or alloys thereof. The length of guide-wire device 100 from the distal portion of tip 108 to the proximal portion of middle inner shaft 204 may be, for example, approximately 25-40 cm. Further, tip 108 may consist of a kink-resistant material, such as, nitinol. It is contemplated that the use of such kink-resistant materials can ensure that the distal portion of guide-wire device 100, which will reside outside the distal end of the endoscope (i.e., the working portion of guide-wire device 100), will be sufficiently robust to withstand bending and manipulation through the elevator portion of a duodenoscope in a typical ERCP procedure.

Proximal inner shaft 300 may be a tubular structure comprised of at least one metal. The metal of proximal inner shaft 300 may be, but not limited to, stainless steel, nickel, titanium cobalt, or alloys. In an embodiment, proximal inner shaft is manufactured from stainless steel or derivatives thereof.

Middle inner shaft 204 is attached to proximal inner shaft 300 via adhesive bonding, welding, laser welding, soldering or other metal joining techniques. Middle inner shaft 204 and proximal inner shaft 300 may be manufactured from a range of rigid or semi-rigid polymer materials. Such materials may be, for example, poly-ether-keytone, poly-ether-ether-keytone, polyamide, polyimide, poly-ether-amide or copolymers thereof, polyethylene, polyurethane, polycarbonate, polystyrene and/or derivatives of all the aforementioned polymer materials.

Middle inner shaft 204 and/or proximal inner shaft 300 may contain a reinforcing member such as, stainless steel braid wire or helically wound stainless steel coil. The inclusion of at least one reinforcing member may aid in improving the kink resistance of middle inner shaft 204 and proximal inner shaft 300, which would be beneficial for clinical procedure enhancement.

The proximal portion of proximal inner shaft 300 includes an inflation gasket 302 to facilitate the inflation and deflation of balloon 102. Inflation gasket 302 may be attached to the inner lumen of proximal inner shaft 300 via mechanical locking and or adhesive bonding. Inflation gasket 302 may be manufactured from a low durometer material such as, silicone, latex, polyurethane, rubber or derivatives thereof, with a typical shore hardness in the range of 15-120 Shore A.

Inflation hub 104 of guide-wire device 100 may be comprised of an injection molded housing, which encapsulates a needle valve with a thru-bore diameter. The proximal portion of inflation hub 104 may include a luer thread element, which is used to attach the component to an external inflation mechanism, such as a syringe or inflation pump. Inflation hub 104 may further include a wire seal element gasket 304 or an o-ring component, which serves to maintain an airtight seal around the proximal end of guide-wire device 100 during the inflation of balloon 102.

In an embodiment of the present disclosure, inflation hub 104 consists of a round tipped needle 306, which protrudes through inflation gasket 302 during inflation. During inflation, needle 306 traverses inflation gasket 302 and media (air or saline) is injected through inflation gasket 302 to inflate balloon 102. Once the required amount of inflation has been achieved, the inflation is ceased, inflation hub 104 and the external inflation means are removed, and balloon 102 remains inflated. In another embodiment, inflation of balloon 102 may be achieved through the inclusion of external or internal threads in inflation hub 104 and guide-wire body 106. To deflate balloon 102, inflation hub 104 is re-attached to guide-wire housing 106, needle 306 is inserted into inflation gasket 302, and balloon 102 is allowed to deflate. Deflation of balloon 102 may be allowed to occur naturally. In another embodiment, a syringe may be attached to inflation hub 104, and a vacuum may be applied to deflate balloon 102.

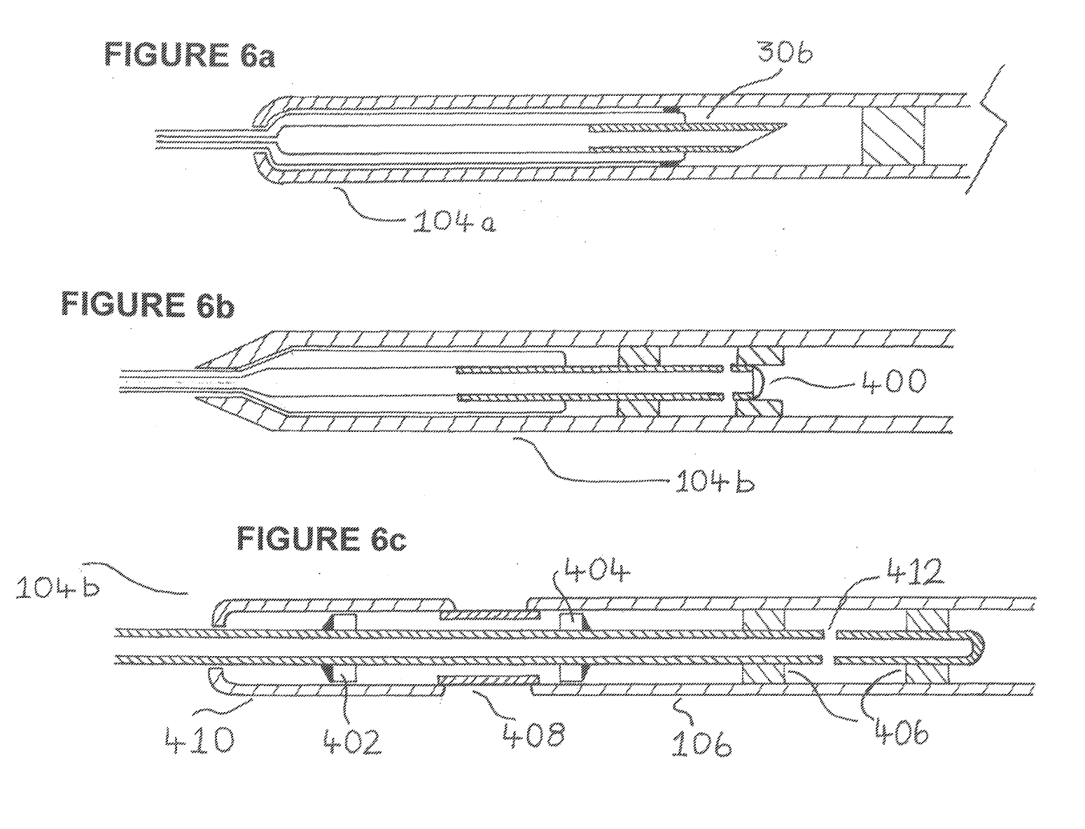

Referring to FIG. 6a, a perspective view of an embodiment of an inflation hub 104a is presented. Inflation hub 104a operates similar to the functionality of a trombone. Inflation hub 104a may initially be advanced through inflation gasket 302. Once balloon 102 has reached a desired inflation diameter, needle 306 may be retracted. Inflation gasket 302 can subsequently collapse to provide an air and/or fluid type seal, which avoids inadvertent deflation of balloon 102. In order to deflate balloon 102, inflation hub 104a may be re-attached to guide-wire housing 106 and advanced through gasket 302. As a result, balloon 102 can deflate as air and/or fluid is extracted from guide-wire device 100 via the use of a syringe or other inflation and deflation means. In the present embodiment, inflation hub 104 may be detached from guide-wire jacket 106 or may remain attached to guide-wire jacket 106. In the latter instance, it is contemplated that the profile (outer diameter) of inflation hub 104 is less than or equal to the outer diameter of guide-wire jacket 106 to facilitate the exchange of ancillary devices thereover.

Referring to FIGS. 6b and 6c, a perspective view of an embodiment of an inflation hub 104b is presented. Inflation hub 104b is comprised of a needle 400, which is mounted with a proximal stop 402 and a distal stop 404. Inflation hub 104b may move forward and backward through at least one internal gasket 406, which is bonded to the internal diameter of guide-wire body 106. At least one gasket 406 may be manufactured from a low durometer material such as, silicone, latex, polyurethane, rubber or derivatives thereof. Gasket 406 may have a typical shore hardness in the range of 15-120 Shore A. In an embodiment of the present disclosure, gasket 406 is comprised of a material that has sufficient hysteresis that can allow gasket 406 to recover its original shape once inflation hub 104b has been removed.

In the present embodiment, a user may advance inflation hub 104b, which contains a radiused thru-bore lumen, through at least one gasket 406 until proximal stop 402 engages with the proximal end of an internal spacer 408. Internal spacer 408 connects an inflation fitting 410 to the proximal body of guide-wire housing 106. Inflation hub 104b may further incorporate at least one circular or elliptical hole 412 immediately proximal to the extreme distal end of needle 400. Hole 412 preferably serves as means to inject air and/or fluid media into guide-wire device 100 and to inflate balloon 102.

In an ERCP procedure, once the desired inflation of balloon 102 has been achieved, inflation hub 104b may be retracted by sliding inflation fitting 410 proximally over internal spacer 408 until distal stop 404 engages with the distal end of internal spacer 408. Subsequently, gasket 406 collapses to provide an air and/or fluid type seal to avoid the inadvertent deflation of balloon 102. To deflate balloon 102, inflation hub 104b is advanced through gasket 406 and balloon 102 is allowed to deflate as air and/or fluid is extracted via the use of a syringe or other inflation and deflation means. In this instance, inflation fitting 410, internal spacer 408, and inflation hub 104b remain attached to the proximal portion of guide-wire housing 106. In an embodiment of the present disclosure, the profile (outer diameter) of inflation fitting 410 is less than or equal to the outer diameter of guide-wire housing 106 to facilitate the exchange of ancillary devices thereover.

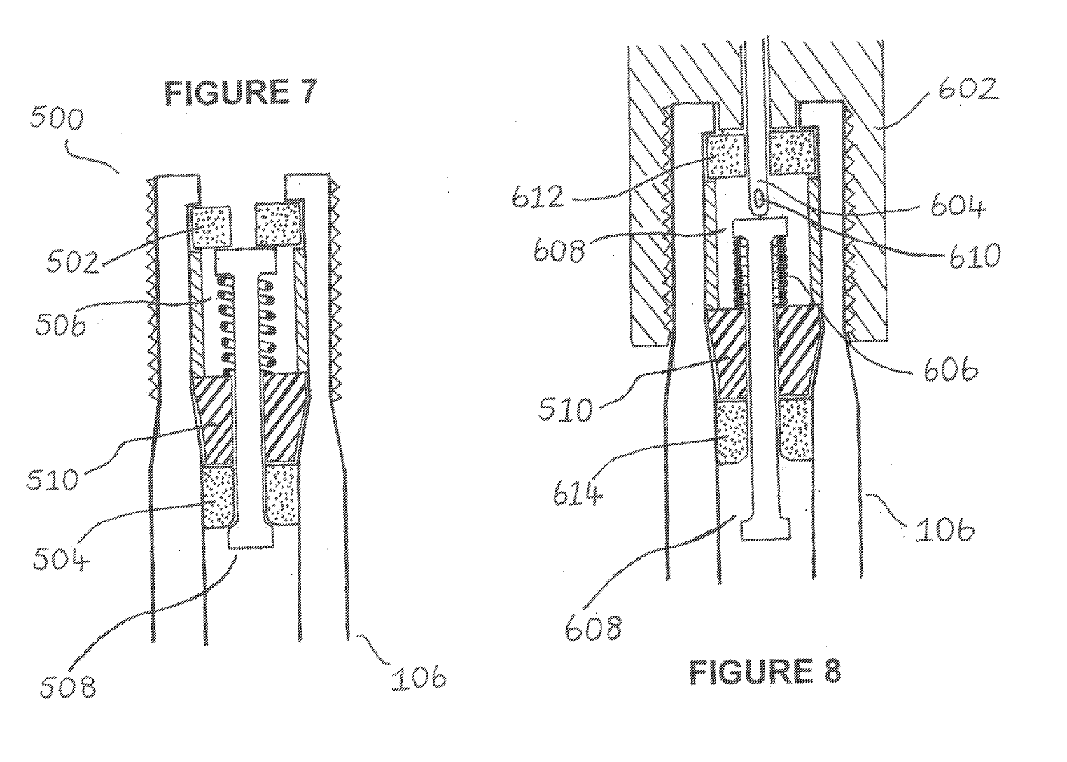

Referring to FIG. 7, a perspective view of an embodiment of an inflation check valve 500 is presented. In an embodiment of the present disclosure, the inflation of balloon 102 is achieved via the use of a “piston type” inflation check valve 500, which is incorporated into the proximal end of guide-wire housing 106. Inflation check valve 500 is presented in a closed position.

Inflation check valve 500 is generally comprised of a proximal gasket 502, a distal gasket 504 a compression spring 506, and a piston valve 508. Proximal gasket 502 and distal gasket 504 may be bonded to the internal diameter of guide-wire housing 106 via the use of adhesive bonding or other techniques. Additionally, proximal gasket 502 and distal gasket 504 may be manufactured from, for example, a low durometer material such as silicone, latex, polyurethane, rubber or derivatives thereof. Further, proximal gasket 502 and distal gasket 504 may have a typical shore hardness in the range of 15-120 Shore A. In an embodiment, proximal gasket 502 and distal gasket 504 are comprised of a material which is deformable and recoverable.

In the “closed” position, compression spring 506 abuts a spring spacer 710, which is locked in position to the body of guide-wire housing 106 at the proximal end. Compression spring 506 may engage the proximal most flange of piston valve 508, which prevents the proximal end of piston valve 508 from abutting proximal gasket 502. The applied force of compression spring 506 in the proximal direction may ensure that the distal end of piston valve 508 can seat tightly against distal gasket 504. In this way, a preferably airtight seal may be achieved between piston valve 508, proximal gasket 502, and distal gasket 504. In an embodiment of the present disclosure, the proximal outer diameter end of guide-wire housing 106 is threaded to facilitate the attachment of an external inflation means to inflate balloon 102.

Referring to FIG. 8, a perspective view of inflation check valve 500 is presented. In the present embodiment, an inflation hub 602 with a needle 604, which is closed-ended and radiused at its distal end, is attached to a proximal portion of guide-wire housing 106 to introduce air and/or fluid media into guide-wire device 100.

In an embodiment, inflation 106 may be secured to guide-wire housing 602 by engaging screw threads, which are located on the proximal portion of guide-wire housing 106, and tightening in place. As inflation hub 602 and needle 604 are tightened onto guide-wire housing 106, a spring 606 becomes compressed as a piston valve 608 pushes forward. Needle 604 can incorporate at least one circular or elliptical holes 610, which may be immediately proximal to the extreme distal end of needle 604. Once inflation hub 602 has been securely fastened in position, inflation media may be injected alongside the longitudinal axis of piston valve 608 to inflate balloon 102. Once a desired state of inflation has been achieved, a user may detach inflation hub 602 from guide-wire device 100. Upon detaching inflation hub 602, piston valve 608 may retract under the compressive force of spring 606. Piston valve 608 may subsequently seal under the force of spring 804 against a proximal gasket 612 and a distal gasket 614. As a result, balloon 102 may maintain its inflated diameter.

In another embodiment of the present disclosure, a user may deflate balloon 102 by attaching inflation hub 602 to the proximal end of guide-wire housing 106. Upon attaching inflation hub 602, needle 604 may cause piston valve 608 to advance forward as air and/or fluid is extracted from guide-wire device 100 via the use of a syringe or other inflation and/or deflation means.

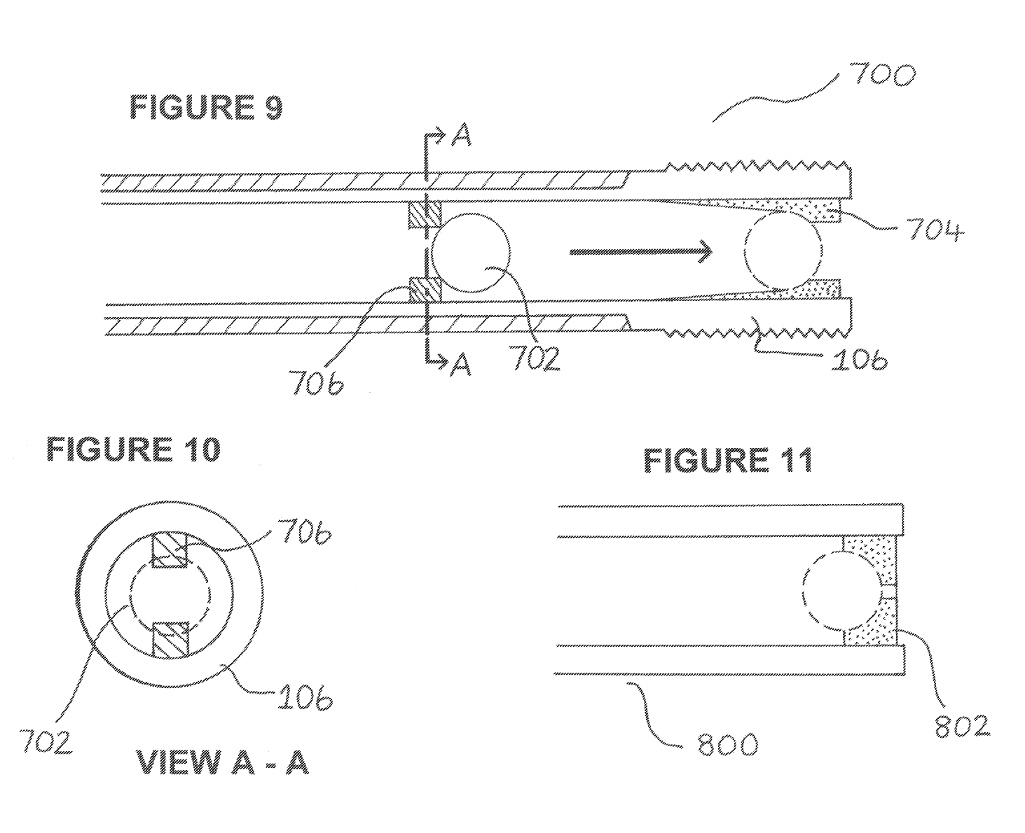

Referring to FIGS. 9 and 10, a perspective and cross-sectional view of an embodiment of an inflation check valve 700 is presented. Inflation check valve 700 is comprised of a check valve ball 702 in contrast to valve piston 608 of inflation check valve 600 of FIG. 8. Check valve ball 702 may be adopted to ensure that an air and/or fluid tight seal is achieved within inflation check valve 700 to maintain the inflation diameter of balloon 102.

In an embodiment of the present disclosure, a user may attach inflation hub 104 to the proximal end of guide-wire housing 106. As inflation media is injected into guide-wire device 100, internal pressure moves check valve ball 702 forward from a proximal sealing gasket 704 towards a check valve stop 706. Check valve stop 706 prevents check valve ball 702 from moving distally along the internal lumen of guide-wire housing 106. In the present embodiment, the injected inflation media may seamlessly pass over check valve ball 702 to facilitate the inflation of balloon 102. Once balloon 102 has been inflated, a user may detach inflation hub 104 from the proximal end of guide-wire housing 106 and check valve ball 702 may subsequently reseat under pressure against sealing gasket 706. A user may deflate balloon 102 by reattaching inflation hub 106 to the proximal end of guide-wire housing 106. Upon reattaching inflation hub 106, check valve ball 702 is displaced from sealing gasket 706 as air and/or fluid is extracted from guide-wire device 100 via the use of a syringe or other inflation/deflation means.

Referring to FIG. 11, a perspective view of another embodiment of an inflation check valve 800 is presented. In the present embodiment, inflation check valve 800 includes a sealing gasket 802, which covers the entire cross sectional area of the inner lumen of guide-wire housing 106. It is contemplated that sealing gasket 802 can promote an improved leak-proof seal as inflation media in injected into guide-wire device 100.

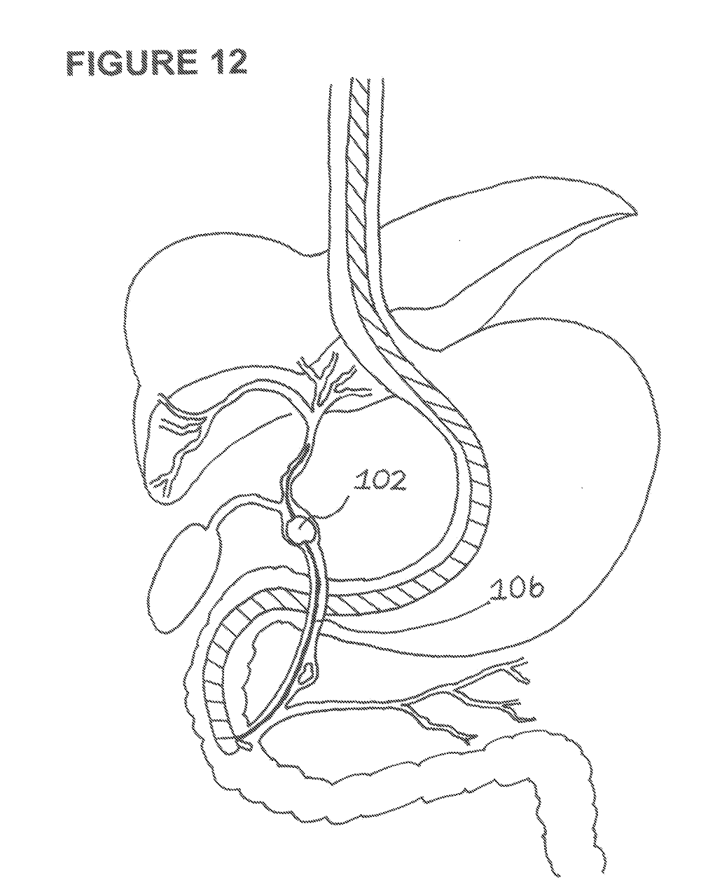

Referring to FIG. 12, an embodiment of guide-wire device 100 utilized in an example field of use, namely the pancreaticobiliary system, is presented. In the present embodiment, a user may utilize an endoscope (duodenoscope), which is positioned trans-orally, by advancing the endoscope down a patient's esophagus, through the stomach and around the duodenum. The working channel exit of the endoscope may be positioned at the duodenum. The papilla can be subsequently cannulated with a sphinctertome or papillatome. Contrast media may be injected into the patient's biliary tree once cannulation is achieved. The contrast media illuminates the patient's ducts for radiographic examination. The user may then position guide-wire housing 106 into the patient's common bile duct to advance and/or retract balloon 102 to remove stones and debris from the common and/or intra-hepatic ducts. The act of removal may be achieved via a sweeping motion across the patient's ampulla of vater while maintaining the inflation pressure of balloon 102.

In the present embodiment, it is contemplated that catheters and ancillary procedural devices may be exchanged over the guide-wire scheme without the need to remove guide-wire device 100, while maintaining access and position of guide-wire housing 106 in the patient's pancreaticobiliary system. As the ERCP procedure is being conducted, balloon 102 may be used to withdraw stones or foreign debris from the biliary ductal system. The removal of such foreign bodies may be achieved by sweeping balloon 102 down the common bile duct and pulling balloon 102 through the papilla at the entrance to the biliary ductal system. The removed or swept elements are then allowed to fall freely into the patient's duodenum where it may be removed via natural body excretion. Tip 108 remains across the papilla during the sweeping of the biliary ductal system, maintaining access to the biliary ductal system with guide-wire device 100.

It will be understood that various modifications may be made to the embodiments disclosed herein. Therefore, the above description should not be construed as limiting, but merely as exemplifications of the various embodiments of the present disclosure. Those skilled in the art will envision other modifications within the scope and spirit of the claims appended hereto.

What is claimed is:

1. A guide-wire device comprising:
a guide-wire housing having proximal and distal portions;
an inner shaft member disposed within the guide-wire housing, the inner shaft member extending from the proximal portion of the guide-wire housing through the distal portion of the guide-wire housing;
a balloon for use in foreign body extraction, the balloon disposed to the distal portion of the guide-wire housing;
an inflation hub disposed to the proximal portion of the guide-wire housing, the inflation hub including means to inflate and deflate the balloon; and
a flexible tip disposed to the distal portion of the balloon, the flexible tip housing a core comprised of kink-resistant materials.

2. The device of claim 1, wherein the inflation hub is adapted to form a non-permanent coupling with the proximal portion of the guide-wire housing.

3. The device of claim 1, wherein the inflation hub is adapted to form a permanent coupling with the proximal portion of the guide-wire housing.

4. The device of claim 1, wherein the inner shaft member includes a proximal inner shaft, a middle inner shaft, and a distal inner inflation shaft.

5. The device of claim 4, wherein the distal inner inflation shaft includes at least one aperture to facilitate the inflation of the balloon.

6. The device of claim 1, wherein the inner shaft includes at least one inflation gasket to facilitate the inflation and deflation of the balloon.

7. The device of claim 1, wherein the inner housing of the tip is comprised of materials to enhance radiopacity.

8. A guide-wire device comprising:
a guide-wire housing having proximal and distal portions;
an inner shaft member disposed within the guide-wire housing, the inner shaft member extending from the proximal portion of the guide-wire housing through the distal portion of the guide-wire housing;
a balloon for use as an anchoring means to facilitate the exchange of ancillary devices over the guide-wire housing, the balloon disposed to the distal portion of the guide-wire housing;
an inflation hub disposed to the proximal portion of the guide-wire housing, the inflation hub including means to inflate and deflate the balloon; and
a flexible tip disposed to the distal portion of the balloon, the flexible tip housing a core comprised of kink-resistant materials.

9. The device of claim 8, wherein the inflation hub is adapted to form a non-permanent coupling with the proximal portion of the guide-wire housing.

10. The device of claim 8, wherein the inflation hub is adapted to form a permanent coupling with the proximal portion of the guide-wire housing.

11. The device of claim 8, wherein the inner shaft member includes a proximal inner shaft, a middle inner shaft, and a distal inner inflation shaft.

12. The device of claim 11, wherein the distal inner inflation shaft includes at least one aperture to facilitate the inflation of the balloon.

13. The device of claim 8, wherein the inner shaft includes at least one inflation gasket to facilitate the inflation and deflation of the balloon.

14. The device of claim 8, wherein the inner housing of the tip is comprised of materials to enhance radiopacity.

15. A guide-wire device comprising:
a guide-wire housing having proximal and distal portions;
an inner shaft member disposed within the guide-wire housing, the inner shaft member including a proximal inner shaft, a middle inner shaft, a distal inner inflation shaft, and an inflation check valve, and the inner shaft member extending from the proximal portion of the guide-wire housing through the distal portion of the guide-wire housing;
a balloon for use in foreign body extraction and as an anchoring means to facilitate the exchange of ancillary devices over the guide-wire housing, the balloon disposed to the distal portion of the guide-wire housing;
an inflation hub adapted to form a coupling with the proximal portion of the guide-wire housing, the inflation hub disposed to the proximal portion of the guide-wire housing, and the inflation hub including means to inflate and deflate the balloon; and
a flexible tip disposed to the distal portion of the balloon, the flexible tip housing a core comprised of kink-resistant materials.

16. The device of claim 15, wherein the inner inflation shaft includes at least one aperture to facilitate the inflation of the balloon.

17. The device of claim 15, wherein the inflation check valve is comprised of a proximal gasket, a distal gasket, a spring, a spring spacer, and a valve piston.

18. The device of claim 15, wherein the inflation check valve is comprised of a proximal gasket, a distal gasket, and a check valve ball.

19. The device of claim 15, wherein the inner housing of the tip is comprised of materials to enhance radiopacity.
